(19) United States
(12) Patent Application Publication
(10) Pub. No.: US 2017/0136501 A1
(43) Pub. Date: May 18, 2017

(54) SYSTEMS AND METHODS FOR CLEANING AND DISINFECTING ELECTRONIC SCREENS AND OTHER HARD SURFACES

(71) Applicant: Martin Rifkin, Vancouver, WA (US)

(72) Inventor: Martin Rifkin, Vancouver, WA (US)

(21) Appl. No.: 14/940,878

(22) Filed: Nov. 13, 2015

(51) Int. Cl. B08B 1/00; C11D 3/48; A61L 2/18; B08B 3/08; B05B 11/00

(52) U.S. Cl. CPC B08B 1/006; B08B 3/08; B05B 11/30; A61L 2/18; C11D 3/48

(57) ABSTRACT

A system for cleaning and disinfecting a hard surface includes housing having a base and a front cover panel, an atomizer nozzle, a fluid reservoir housed within the base, and a volume of fluid contained in the fluid reservoir. The fluid may contain colloidal silver. The base of the housing may include a convex rear shell and a first flat face. The front cover panel may include a convex front shell and a complimentary second flat face. The second flat face of the front cover panel may be slidably affixed to first flat face of the base. The front cover panel may be configured to slide, relative to the base, from a first position to a second position, thereby activating the atomizer nozzle and spraying fine liquid droplets containing silver onto the hard surface. At least some bacteria on the hard surface may be killed via the silver.

### BACKGROUND

The present disclosure relates generally to systems and methods of cleaning and disinfecting electronic screens. In particular, systems and methods of cleaning and disinfecting electronic screens using colloidal silver are described.

Electronic devices (e.g., cell phones, tablets, laptop computers, etc.) are increasingly becoming part of everyday life. Thus, human users are constantly touching their electronic devices, especially the touch screens of such devices, thereby transferring dirt, fingerprints, grime and/or bacteria from their hands onto their devices. Such bacteria may include E. coli, Methicillin-Resistant Staphylococcus Aureus (MRSA) and/or the flu, among others.

Electronic devices not only collect dirt, fingerprints, grime, and/or bacteria, but they may also breed bacteria due to their relatively warm operating temperatures and being stored in warm dark spaces like pockets, purses and cases. It is estimated that the average cell phone has eighteen times more harmful bacteria than the handle in a public restroom. Despite all this, while most people wash their hands regularly, few clean their electronic devices.

The excess bacteria on electronic devices can negatively affect a user's immune system and overall health, potentially compromising the user's overall ability to fight off infection. Bacteria on phones may also cause acne. When a user presses his or her cheek and/or chin against their phone, bacteria may transfer from the phone onto the skin and into the pores of the user.

Conventional means for cleaning and sanitizing surfaces of are not entirely satisfactory when applied to electronic devices. For example, soaking or saturating with sanitizing liquid may damage the delicate electronics often found in electronic devices.

Thus, there exists a need for systems and methods of cleaning and disinfecting screens of electronic devices and other hard surfaces. Examples of new and useful systems for cleaning and disinfecting hard surfaces, including screens, relevant to the needs existing in the field are discussed below.

### SUMMARY

In one embodiment, a system for cleaning and disinfecting a hard surface includes a housing having a base and a front cover panel, an atomizer nozzle, a fluid reservoir housed within the base, and a volume of fluid contained in the fluid reservoir. The fluid may contain colloidal silver.

The base of the housing may include a convex rear shell and a first flat face. The front cover panel may include a convex front shell and a complimentary second flat face. The second flat face of the front cover panel may be slidably affixed to first flat face of the base. In this regard, the front cover panel may be configured to slide, relative to the base, from a first position to a second position.

In some embodiments, the system may include a power source in selective electrical communication with the atomizer nozzle. In some embodiments, the electrical switch may be triggered by sliding the front cover panel to the second position. In some embodiments, the electrical switch may be configured to control the electrical communication between the power source and the atomizer nozzle.

In one embodiment, a method includes pointing a cleaning and disinfecting device at a hard surface. The hard surface may have bacteria thereon. The cleaning and disinfecting device may include a housing, an atomizer nozzle disposed on the housing, a fluid reservoir housed within the housing and in fluid connection with the atomizer nozzle, and a volume of fluid contained in the fluid reservoir. The fluid may include colloidal silver. The method may further include the steps of activating the atomizer nozzle, and pumping, in response to the activating step, at least some of the fluid through the atomizer nozzle, thereby creating fine droplets of fluid. The fine droplets may be sprayed onto the hard surface. In some embodiments, the fine droplets contain silver. Thus, at least some bacteria on the hard surface may be killed via the silver in the droplets. Further, the process may clean dirt, fingerprints, and/or grime from the hard surface. In some embodiments, the hard surface may be a touch screen of an electronic device.

### BRIEF DESCRIPTION OF THE DRAWINGS

FIG. 1 is a front elevation view of one embodiment of a cleaning and disinfecting system showing the front cover panel of the cleaning and disinfecting system in a first position.

FIG. 2 is a front elevation view of the disinfecting system of FIG. 1 showing the front cover panel of the cleaning and disinfecting system in a second position.

FIG. 3 is a side elevation view of the cleaning and disinfecting system of FIG. 1 showing the front cover panel in the first position.

FIG. 4 is a side elevation view of the cleaning and disinfecting system of FIG. 1 showing the front cover panel in the second position.

FIG. 5 is a rear elevation view of the cleaning and disinfecting system of FIG. 1 showing a fluid reservoir of the disinfecting system after being removed from the housing of the cleaning and disinfecting system.

FIG. 6 is a side elevation view of the cleaning and disinfecting system of FIG. 1 showing the hatch door of the cleaning and disinfecting system in an open position and the contents of the hatch being replaced.

FIG. 7 is a rear elevation view of the cleaning and disinfecting system of FIG. 1 showing the hatch door of the cleaning and disinfecting system in an open position.

### DETAILED DESCRIPTION

The disclosed systems and methods for cleaning and disinfecting electronic screens will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various systems and methods for cleaning and disinfecting electronic screens are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

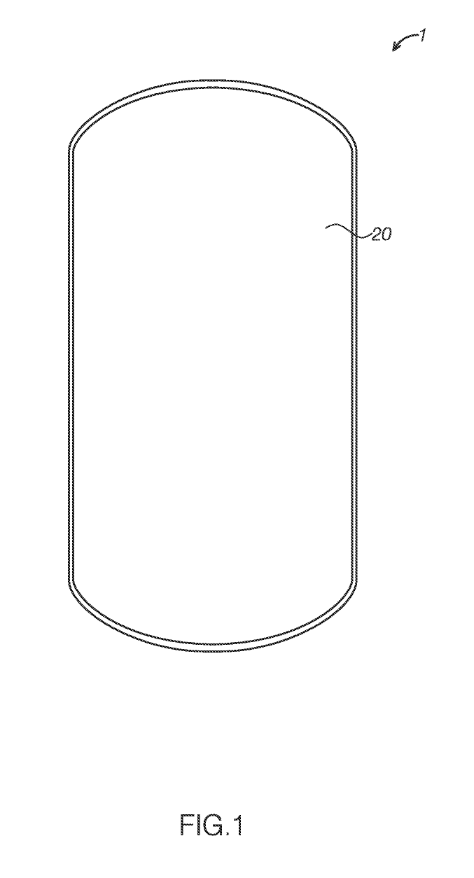

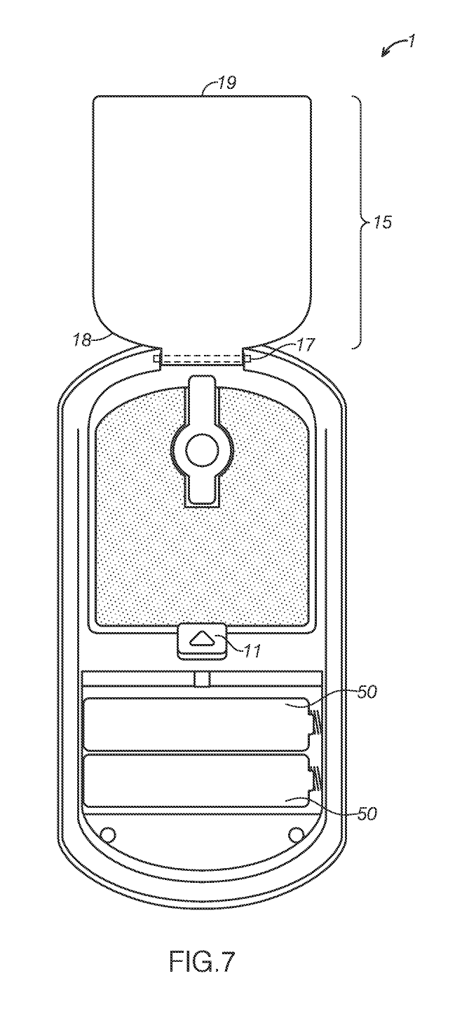

With reference to FIGS. 1-7, a first example of a system for cleaning and disinfecting electronic screens and other hard surfaces, cleaning and disinfecting system 1, will now be described. Cleaning and disinfecting system 1 functions to kill at bacteria on the surface(s) of one or more hard surfaces via colloidal silver. The reader will appreciate from the figures and description below that system addresses the need for a method to clean and sanitize electronic devices.

Cleaning and disinfecting system 1 includes a housing having a base 10 and a front cover panel 20, an atomizer nozzle 30, a fluid reservoir 42 housed within the base, and a volume of fluid 42 contained in the fluid reservoir. Cleaning and disinfecting system 1 may include a cleaning cloth 80. In some embodiments, cleaning cloth 80 may include silver particles imbedded throughout the cloth.

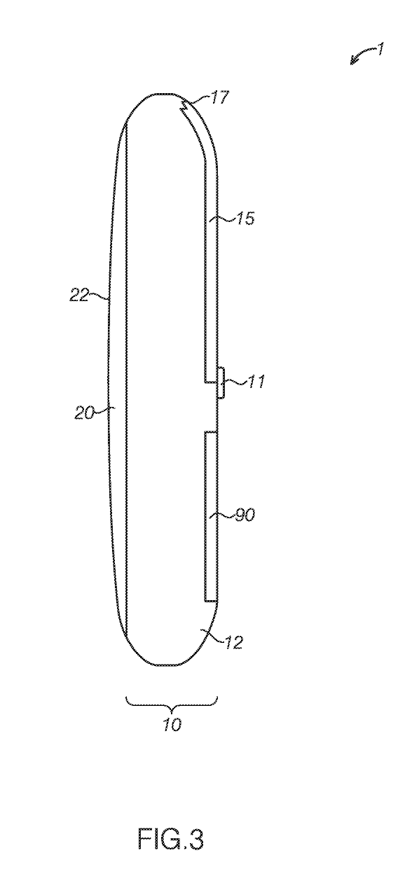

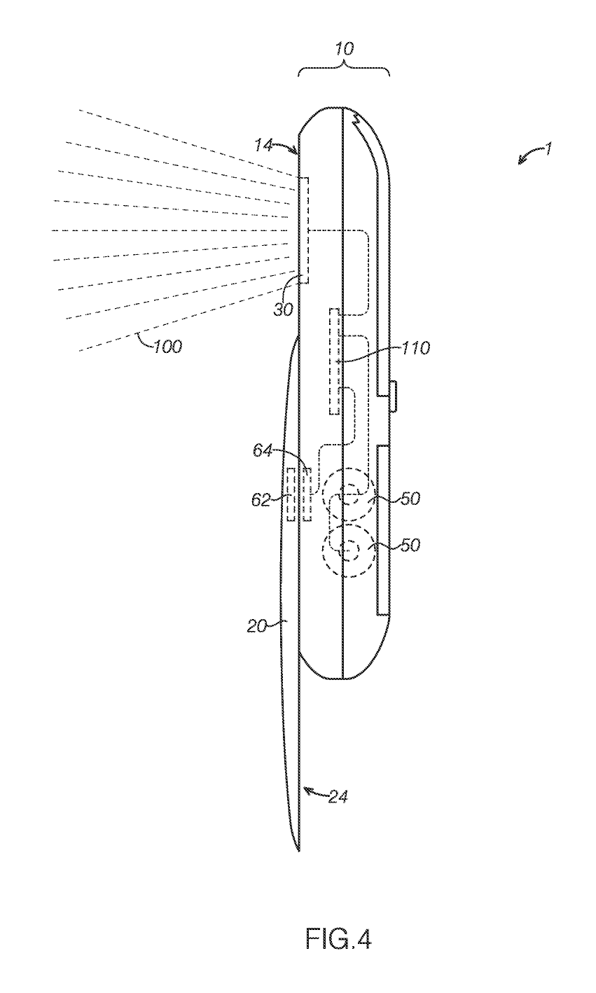

As can be seen in FIGS. 3-4, the base 10 of the housing includes a convex rear shell 12, and a first flat face 14. The front cover panel 20 of the housing includes a convex front shell 22 and a complimentary second flat face 24. Second flat face 24 may be slidably affixed to first flat face 14. In this regard, front cover panel 20 may be configured to slide from a first position, as shown in FIG. 3, to a second position, as shown in FIG. 4. In some embodiments, first flat face 14 may include one or more rails and second flat face 24 may include one or more complementary clasping devices slidably affixed to the rails. In some embodiments, disinfecting system may also include one or more springs. The one or more springs may be attached to the rails and/or clasping devices to lock the front cover panel 20 in the first position and/or the second position.

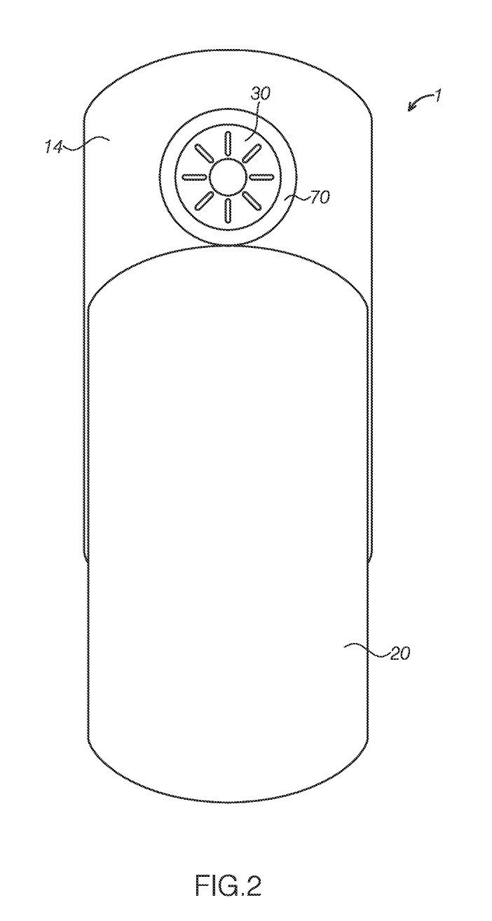

As shown in FIGS. 2 and 4, atomizer nozzle 30 may be disposed on first flat face 14. In this regard, in some embodiments, when front cover panel 20 is in the first position, atomizer nozzle 30 may be covered by front cover panel 20. Furthermore, in some embodiments, when front cover panel is in the second position, atomizer nozzle 30 may be exposed. Thus, when cleaning and disinfecting system 1 is not in use, front cover panel 20 may function to protect atomizer nozzle from dust and/or damage.

Atomizer nozzle 30 may be configured to produce a mist of fine droplets of fluid. For example, in some embodiments, atomizer nozzle may include an electrically powered pumping device to force liquid through the nozzle. In some embodiments, the fine mist may facilitate full coverage of the surface to be cleaned and disinfected without saturating or submerging the surface. In this regard, the fine mist may allow for cleaning and disinfecting screens of electronic devices which may be too delicate to be submerged or saturated with liquid.

In some embodiments, atomizer nozzle 30 may be configured to produce droplets of fluid having a volume mean droplet diameter of 10 to 250 microns. In some embodiments, atomizer nozzle 30 may be configured to produce droplets of fluid having a volume mean droplet diameter of 10 to 200 microns. In some embodiments, atomizer nozzle 30 may be configured to produce droplets of fluid having a volume mean droplet diameter of 10 to 150 microns. In some embodiments, atomizer nozzle 30 may be configured to produce droplets of fluid having a volume mean droplet diameter of 10 to 100 microns.

In some embodiments, atomizer nozzle 30 may be configured to produce droplets of fluid having a volume mean droplet diameter of 25 to 250 microns. In some embodiments, atomizer nozzle 30 may be configured to produce droplets of fluid having a volume mean droplet diameter of 50 to 250 microns. In some embodiments, atomizer nozzle 30 may be configured to produce droplets of fluid having a volume mean droplet diameter of 100 to 250 microns. In some embodiments, atomizer nozzle 30 may be configured to produce droplets of fluid having a volume mean droplet diameter of 150 to 250 microns.

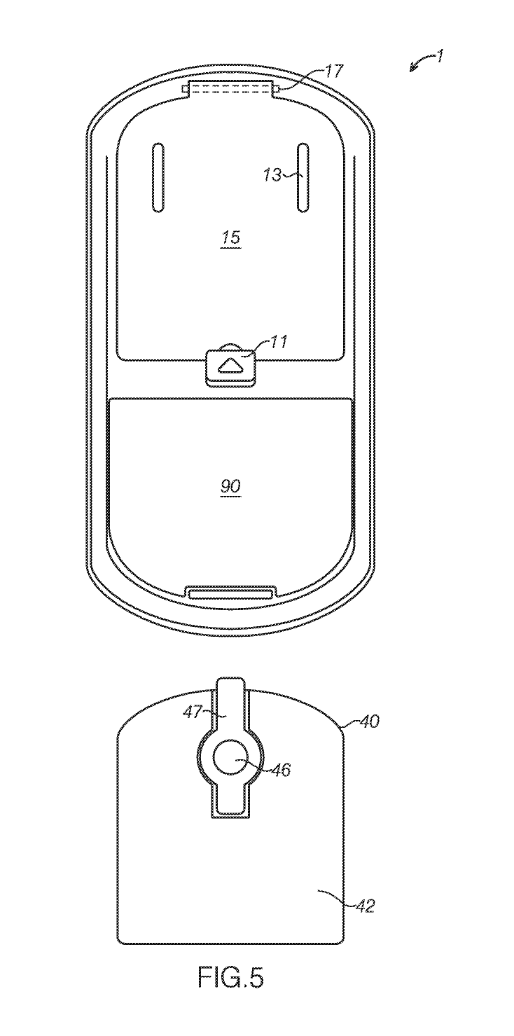

As shown in FIGS. 5-7, cleaning and disinfecting system 1 may include fluid reservoir 40 housed within the base. Fluid reservoir may contain a fluid 42. Fluid reservoir 40 may be in fluid communication with atomizer nozzle 30. In this regard, fluid 42 may flow from the fluid reservoir 40 through the atomizer nozzle 30 in order to produce the liquid droplets.

Fluid 42 may include colloidal silver. In one embodiment, the fluid may contain 5 to 100 ppm (parts per million) colloidal silver. In one embodiment, the fluid may contain 10 to 75 ppm (parts per million) colloidal silver. In one embodiment, the fluid may contain 15 to 50 ppm (parts per million) colloidal silver. In one embodiment, the fluid may contain 20 to 40 ppm (parts per million) colloidal silver.

In some embodiments, fluid reservoir 40 includes a removable plug 46. In this regard, fluid reservoir 40 may include a socket configured to receive removable plug 46 and thereby form a fluid-tight seal. In some embodiments, fluid reservoir 40 may be refilled via the socket. Thus, in some embodiments, fluid reservoir 40 may be a refillable fluid reservoir.

In one embodiment, the fluid reservoir may be replaceable and/or disposable. In this regard, when the fluid reservoir becomes empty, the entire fluid reservoir may be replaced with a new fluid reservoir.

In some embodiments, cleaning and disinfecting system 1 may include a flexible strap 47. Flexible strap 47 may have a first end 48 and a second end 49. The first end 48 of flexible strap 47 may be affixed to the removable plug 46. The second end 49 of the flexible strap 47 may be affixed to the base 10. In this regard, the flexible strap may serve to hold fluid reservoir 40 in place within the housing.

In some embodiments, cleaning and disinfecting system 1 may include a power source in electrical communication with the atomizer nozzle 30. In some embodiments the power source may comprise batteries 50. Batteries 50 may include any suitable batteries, including AA, AAA, C, D, 12 volt, as well as coin cell batteries, among others. In some embodiments, the power source may be in selective electrical communication with the atomizer nozzle 30. For example, in some embodiments, cleaning and disinfecting system 1 may include an electrical switch configured to control the electrical communication between the power source and the atomizer nozzle.

In some embodiments, the power source may include one or more rechargeable batteries. In this regard, the system may include a charging cord or charging station to recharge the rechargeable batteries. In some embodiments, the cleaning and disinfecting device may include a USB port, with the charging cord being configured to charge the batteries via the USB port.

In some embodiments, the electrical switch may comprise a magnet 62 disposed within front cover panel 20, magnetic sensor 64 disposed with base 10, a circuit board 110 disposed with base 10. When magnetic sensor 64 senses magnet 62 in close proximity, magnetic sensor 64 may send an activate signal to circuit board 110. Circuit board 110 may then open the electrical communication between batteries 50 and atomizer nozzle 30, thereby activating atomizer nozzle 30 and producing liquid droplets.

As shown in the embodiment of FIG. 4, sliding the front cover panel to the second position places magnet 62 in close proximity to magnetic sensor 64. In this regard, in some embodiments, sliding the front cover panel to the second position activates the atomizer nozzle 30.

Furthermore, in some embodiments, when magnetic sensor 64 does not sense magnet 62 in close proximity, magnetic sensor 64 may send a deactivate signal to circuit board 110. Circuit board 110 may then close the electrical communication between batteries 50 and atomizer nozzle 30, thereby deactivating atomizer nozzle 30. In this regard, in some embodiments, sliding the front cover panel back to the first position may deactivate the atomizer nozzle.

In some embodiment, the system 1 may be configured to spray a predetermined volume of fluid 42. For example, in some embodiments, circuit board 110 may include a timer (not pictured). In this regard, activating the atomizer nozzle 30 may initiate the timer. When the timer reaches a predetermined spray duration, the timer may send a deactivate signal to circuit board 110. Circuit board 110 may then deactivate atomizer nozzle 30. The predetermined spray duration may correspond to a predetermined volume of fluid 42.

In one embodiment the system may be configured to spray a predetermined volume of fluid of 0.01 to 10 mL. In one embodiment the system may be configured to spray a predetermined volume of fluid of 0.05 to 5 mL. In one embodiment the system may be configured to spray a predetermined volume of fluid of 0.1 to 1 mL. In one embodiment the system may be configured to spray a predetermined volume of fluid of 0.15 to 0.75 mL. In one embodiment the system may be configured to spray a predetermined volume of fluid of 0.2 to 0.6 mL.

In some embodiments, the cleaning and disinfecting system 1 may include a hatch 15 located in convex rear shell 12 of base 10. The hatch 15 may include a hatch door 16 having a first edge 18 and a second edge 19, a hinge 17, and clasp 11. Hatch 15 may be connected to convex rear shell 12 via hinge 17. Specifically, hinge 17 may connect the first edge 18 of hatch door 16 to the convex shell 12. Clasp 11 may be located on the second edge 19 of hatch door 16, thus allowing the hatch door 16 to swing open, via hinge 17, or be locked closed.

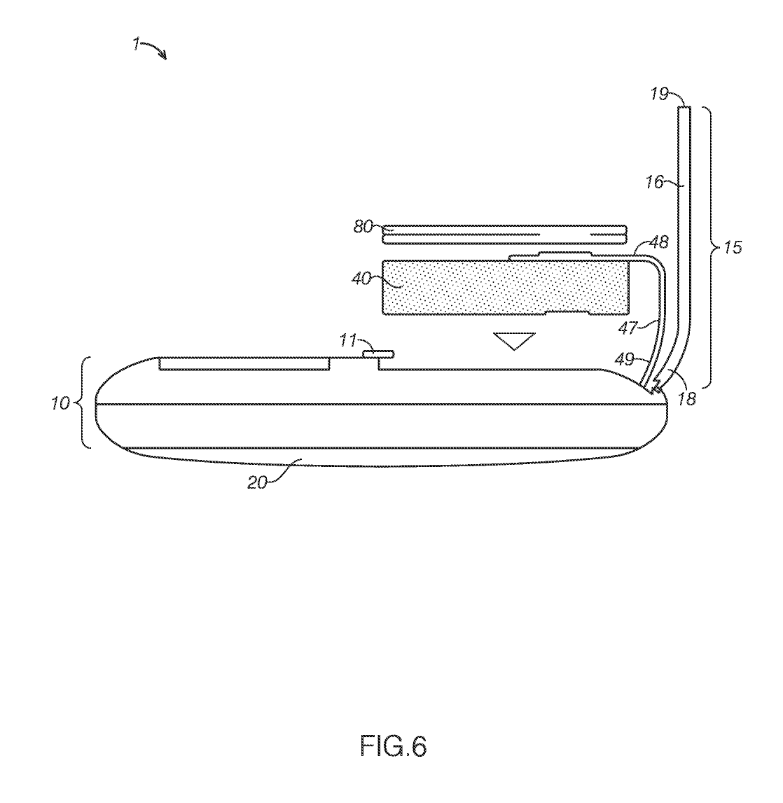

As shown in FIG. 6, hatch 15 may be configured to provide access to fluid reservoir 40. Thus, hatch 15 may facilitate refilling of fluid reservoir 40.

In some embodiments, hatch 15 may also hold a cleaning cloth 80. Cleaning cloth 80 may comprise particles of silver dispersed throughout the fabric of the cloth. Thus, cleaning cloth 80 may be impregnated with silver. The silver impregnated within the cloth may facilitate the cleaning and killing bacteria on hard surfaces. Furthermore, in some embodiments, the system may include a storage pouch. The storage pouch may be configured to enclose an electronic device. The storage pouch may be impregnated with silver to clean and kill bacteria. Thus, the storage pouch may clean and disinfect the outer surfaces of the electronic device while it is being stored therein.

Hatch door 16 may include a fluid level indication slot 13 formed therein. In some embodiments, a user of cleaning and disinfecting system 1 may visually check the fluid level of fluid within the fluid reservoir via the fluid level indication slot 13. In this regard, fluid reservoir 40 may be made of clear or translucent material.

As shown in FIG. 2, in some embodiments, cleaning and disinfecting system 1 may include a misting indication light 70. Misting indication light 70 may be located proximal atomizing nozzle 30. For example, as can be seen in FIG. 2, misting indication light 70 may encircle atomizing nozzle 30. In some embodiments, misting indication light 70 may comprise one or more light emitting diodes (LEDs).

Misting indication light 70 may function to alert the user that the cleaning and disinfecting system has been activated and the atomizer nozzle is currently producing fine droplets. In this regard, misting indication light 70 may be activated in the same or similar manner to atomizer nozzle. For example, in some embodiments when the front cover panel 20 is slid into the second position, the electrical switch may activate the misting indication light 70 as well as the atomizer nozzle 30.

As shown in FIGS. 3 and 5, in some embodiments, cleaning and disinfecting system 1 may include a battery storage compartment 90 formed in the convex outer shell 12 of base 10. The battery storage compartment may be configured to store the one or more batteries 50.

In one embodiment, a method of cleaning and disinfecting a hard surface includes pointing a cleaning and disinfecting device, such as cleaning and disinfecting system 1, at the hard surface, activating an atomizer nozzle of the cleaning and disinfecting device, and pumping, in response to the activating step, at least some of the fluid through atomizer nozzle, thereby creating fine droplets of fluid. The fine droplets may be sprayed onto the hard surface. In some embodiments, the fine droplets contain colloidal silver. Thus, at least some bacteria on the hard surface may be killed via the silver in the droplets. Further, the process may clean dirt, fingerprints, and/or grime from the hard surface.

In one embodiment, the hard surface may have at least some S. aureus, E. coli, and/or S. typhimurium bacteria disposed thereon. In one embodiment at least 80% of the S. aureus bacteria is killed via the silver in the droplets. In one embodiment at least 85% of the S. aureus bacteria is killed via the silver in the droplets. In one embodiment at least 90% of the S. aureus bacteria is killed via the silver in the droplets. In one embodiment at least 95% of the S. aureus bacteria is killed via the silver in the droplets. In one embodiment at least 99% of the S. aureus bacteria is killed via the silver in the droplets. In one embodiment at least 99.9% of the S. aureus bacteria is killed via the silver in the droplets. In one embodiment at least 99.99% of the S. aureus bacteria is killed via the silver in the droplets.

In one embodiment at least 80% of the E. coli bacteria is killed via the silver in the droplets. In one embodiment at least 85% of the E. coli bacteria is killed via the silver in the droplets. In one embodiment at least 90% of the E. coli bacteria is killed via the silver in the droplets. In one embodiment at least 95% of the E. coli bacteria is killed via the silver in the droplets. In one embodiment at least 99% of the E. coli bacteria is killed via the silver in the droplets. In one embodiment at least 99.9% of the E. coli bacteria is killed via the silver in the droplets. In one embodiment at least 99.99% of the E. coli bacteria is killed via the silver in the droplets.

In one embodiment at least 80% of the S. typhimurium bacteria is killed via the silver in the droplets. In one embodiment at least 85% of the S. typhimurium bacteria is killed via the silver in the droplets. In one embodiment at least 90% of the S. typhimurium bacteria is killed via the silver in the droplets. In one embodiment at least 95% of the S. typhimurium bacteria is killed via the silver in the droplets. In one embodiment at least 99% of the S. typhimurium bacteria is killed via the silver in the droplets. In one embodiment at least 99.9% of the S. typhimurium bacteria is killed via the silver in the droplets. In one embodiment at least 99.99% of the S. typhimurium bacteria is killed via the silver in the droplets.

In one embodiment, the hard surface is a screen of an electronic device. For example the hard surface may be a touch-sensitive screen of the electronic device.

In some embodiments, the step of activating the atomizer nozzle may include sliding a front cover panel of the cleaning and disinfecting device from a first position to a second position. In other embodiments, the step of activating the atomizer nozzle may include pushing a button and/or flipping a toggle switch, among others.

In some embodiments, the activating step may include energizing the atomizer nozzle via one or more batteries stored in the housing.

In some embodiments, the method may include wiping the fine droplets off the touch screen via a cleaning cloth, such as cleaning cloth 80. As described above, cleaning cloth 80 may comprise silver particles.

In some embodiments, the method may include indicating that the atomizer is creating fine droplets via a misting indication light located on the housing.

In some embodiments, the method may include refilling the fluid reservoir with fluid.

Electronic devices which may be cleaned in accordance with the above described methods and systems may comprise mobile electronic devices, such as smartphones, laptop personal computers, smart glasses, tablet computers, and/or portable music players among others. In some embodiments, electronic devices may include desktop personal computers that employ a touch screen and/or other generally non-portable electronic devices having a touchscreen.

### Example 1

Three different strains of bacteria were treated with 26 ppm (parts per million) liquid colloidal silver solution in accordance with ASTM E2315 testing guidelines. Staphylococcus aureus (S. aureus) ATCC 25923, Escherichia coli (E. coli) ATCC 25922, and Salmonella typhimurium (S. typhimurium) ATCC 14028 bacteria strains were acquired. Each strain was then cultured on tryptic soy agar with 5% sheep blood at 35° C. for 24 hours.

Each cultured strain was suspended in phosphate buffer and standardized to achieve a final concentration of approximately 108 colony-forming units per milliliter (CFU/mL) using spectrophotometry at 590 nm. One milliliter of each standardized suspension was then treated with 0.50 mL or 0.25 mL of 26 ppm colloidal silver solution. Plate counts were performed at time zero, 15 second, 30 second, and 60 second time intervals to achieve viable bacterial counts. Each experiment was replicated three times. Averages for the three replicate experiments were calculated at each of the time zero, 15 second, 30 second, and 60 second time intervals. Percent reduction and log reduction calculations were made comparing average viability at 15 seconds, 30 seconds and 60 seconds compared to the average at time zero. The results are shown in Tables 1-3, below.

TABLE 1
0.50 mL COLLOIDAL SILVER SOLUTION AGAINST S. aureus

| | | 15-second | 30-second | 60-second |
|---|---|---|---|---|
| | Time zero | Treatment | Treatment | Treatment |
| | Population | Population | Population | Population |
| Replicate | CFU/mL | CFU/mL | CFU/mL | CFU/mL |
| 1 | 7.40 × 10⁶ | 1.32 × 10⁶ | 1.24 × 10⁶ | 2.30 × 10⁵ |
| 2 | 8.20 × 10⁶ | 1.60 × 10⁶ | 1.40 × 10⁶ | 4.70 × 10⁵ |
| 3 | 8.40 × 10⁶ | 1.60 × 10⁶ | 8.40 × 10⁶ | 3.90 × 10⁵ |
| Average | 8.00 × 10⁶ | 1.51 × 10⁶ | 1.16 × 10⁶ | 3.63 × 10⁵ |

| | | | |
|---|---|---|---|
| Log Reduction | 0.73 | 0.85 | 1.36 |
| % Reduction | 81.20 | 85.39 | 95.51 |

TABLE 2
0.25 mL COLLOIDAL SILVER SOLUTION AGAINST E. coli

| | | 15-second | 30-second | 60-second |
|---|---|---|---|---|
| | Time zero | Treatment | Treatment | Treatment |
| | Population | Population | Population | Population |
| Replicate | CFU/mL | CFU/mL | CFU/mL | CFU/mL |
| 1 | 2.80 × 10⁶ | 1.00 × 10¹ | 1.00 × 10¹ | 1.00 × 10¹ |
| 2 | 3.00 × 10⁶ | 1.00 × 10¹ | 1.00 × 10¹ | 1.00 × 10¹ |
| 3 | 4.50 × 10⁶ | 1.00 × 10¹ | 1.00 × 10¹ | 1.00 × 10¹ |
| Average | 3.43 × 10⁶ | 1.00 × 10¹ | 1.00 × 10¹ | 1.00 × 10¹ |

| | | | |
|---|---|---|---|
| Log Reduction | 5.53 | 5.53 | 5.53 |
| % Reduction | 99.99 | 99.99 | 99.99 |

TABLE 3
0.25 mL COLLOIDAL SILVER SOLUTION AGAINST
S. typhimurium

| | | 15-second | 30-second | 60-second |
|---|---|---|---|---|
| | Time zero | Treatment | Treatment | Treatment |
| | Population | Population | Population | Population |
| Replicate | CFU/mL | CFU/mL | CFU/mL | CFU/mL |
| 1 | 8.30 × 10⁶ | 1.00 × 10⁶ | 1.20 × 10² | 1.00 × 10¹ |
| 2 | 5.20 × 10⁶ | 8.30 × 10⁶ | 2.30 × 10² | 1.00 × 10¹ |
| 3 | 8.20 × 10⁶ | 1.30 × 10⁶ | 8.00 × 10² | 1.00 × 10¹ |
| Average | 7.23 × 10⁶ | 1.04 × 10⁶ | 3.83 × 10² | 1.00 × 10¹ |

| | | | |
|---|---|---|---|
| Log Reduction | 0.84 | 4.40 | 5.85 |
| % Reduction | 85.38 | 99.99 | 99.99 |

As can be seen in Table 1, a log reduction of 1.36 and a % reduction of 95.51 of S. aureus were achieved when the S. aureus samples were treated with 0.5 mL of the colloidal silver solution for 60 seconds. As can be seen in Table 2, a log reduction of 5.53 and a % reduction of 99.99 of E. coli were achieved when the E. coli samples were treated with 0.25 mL of the colloidal silver solution for 15 seconds. As can be seen in Table 3, a log reduction of 4.40 and a % reduction of 99.99 were achieved for S. typhimurium when the S. typhimurium samples were treated with 0.25 Ml of silver solution for 30 seconds.

### Example 2

An E. coli strain of bacteria was treated with 26 ppm (parts per million) liquid colloidal silver solution in accordance with ASTM E2315 testing guidelines. E. coli ATCC 25922 bacteria strain was acquired. The E. coli strain was then cultured on tryptic soy agar with 5% sheep blood at 35° C. for 24 hours.

The cultured bacterial E. coli strain was suspended in phosphate buffer and standardized to achieve a final concentration of approximately 106 colony-forming units per milliliter (CFU/mL) using spectrophotometry at 590 nm. One milliliter of standardized bacterial culture was treated for 10-seconds with a colloidal silver atomizing system in accordance with the above description, followed by wiping with a silver ion-containing cloth. The experiment was replicated three times. Plate counts were performed. Averages for the three replicate experiments were calculated. Percent reduction and log reduction calculations were made comparing average viability after colloidal silver treatment to the average untreated bacterial suspension. The results are shown in Table 4, below.

TABLE 4
ATOMIZED COLLOIDAL SILVER SOLUTION AGAINST E. coli

| | Time zero | 10-second Treatment |
|---|---|---|
| | Population | Population |
| Replicate | CFU/mL | CFU/mL |
| 1 | 6.80 × 10⁷ | 4.00 × 10² |
| 2 | 7.20 × 10⁷ | 1.60 × 10² |
| 3 | 4.50 × 10⁶ | 1.00 × 10¹ |
| Average | 4.82 × 10⁷ | 1.90 × 10² |

| | |
|---|---|
| Log Reduction | 5.51 |
| % Reduction | 99.99 |

As can be seen in table 4, the solution produced detectable log reductions of E. coli bacteria. Specifically, the solution achieved a log reduction of 5.51 and a % reduction of 99.99, for E. coli, when treated with an atomizer for 10-seconds containing silver solution, followed by wiping with a silver ion containing cloth.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A method of cleaning and disinfecting a hard surface having fingerprints, dirt, grime and/or bacteria thereon, the method comprising:
pointing a cleaning and disinfecting device at the hard surface, wherein the cleaning and disinfecting device comprises:
a housing;
an atomizer nozzle disposed on the housing;
a fluid reservoir housed within the housing and in fluid connection with the atomizer nozzle; and
a volume of fluid contained in the fluid reservoir,
wherein the fluid includes colloidal silver;
activating the atomizer nozzle;
pumping, in response to the activating step, at least some of the fluid through atomizer nozzle, thereby creating fine droplets of the fluid;
wherein the fine droplets contain silver;
spraying the fine droplets onto the hard surface;
killing at least 80% of the bacteria on the hard surface, via the silver in the droplets.

2. The method of claim 1, comprising killing at least 90% of the bacteria on the hard surface.

3. The method of claim 1, comprising killing at least 95% of the bacteria on the hard surface.

4. The method of claim 1, wherein the fine droplets contain 15 to 50 ppm colloidal silver.

5. The method of claim 1, wherein the fine droplets contain 20 to 40 ppm colloidal silver.

6. The method of claim 1, wherein the fine droplets have a volume mean droplet diameter of 10 to 250 microns.

7. The method of claim 1, wherein the hard surface is a touch screen of an electronic device.

8. The method of claim 1, comprising:
wiping the fine droplets off the screen via a cloth;
wherein the cloth comprises silver particles.

9. The method of claim 1, wherein the activating comprises energizing the atomizer nozzle via one or more batteries stored in the housing.

10. A system for cleaning and disinfecting a hard surface, the system comprising
a housing having a base and a front cover panel;
wherein the base comprises a convex rear shell and a first flat face;
wherein the front cover panel comprises a convex front shell and a complimentary second flat face;
wherein the second flat face of the front cover panel is slidably affixed to first flat face of the base such that front cover panel is configured to slide, relative to the base, from a first position to a second position;
an atomizer nozzle disposed on the first flat face such that:
when the front cover panel is in the first position, the atomizer nozzle is covered by the front cover panel; and
when the front cover panel is in the second position, the atomizer nozzle is exposed;
a fluid reservoir housed within the base and in fluid communication with the atomizer nozzle;
a volume of fluid contained in the fluid reservoir;
wherein the fluid contains colloidal silver.

11. The system of claim 10, wherein the fluid contains 15 to 50 ppm colloidal silver.

12. The system of claim 10, wherein the fluid contains 20 to 40 ppm colloidal silver.

13. The system of claim 10, wherein the atomizer nozzle is configured to spray fine droplets having a volume mean droplet diameter of 10 to 250 microns.

14. The system of claim 10, comprising
a power source in selective electrical communication with the atomizer nozzle.

15. The system of claim 10, comprising
an electrical switch configured to control the electrical communication between the power source and the atomizer nozzle.

16. The system of claim 15, wherein the electrical switch comprises:
a magnet disposed in the front cover panel; and
a magnetic sensor disposed in the base.

17. The system of claim 16, wherein the atomizer nozzle is activated by sliding the front cover panel to the second position.

18. The system of claim 10, comprising
a hatch in the convex shell of the base;
wherein the hatch is configured to provide access to the fluid reservoir.

19. The system of claim 18, wherein the hatch comprises:
a hatch door;
a hinge connecting a first edge of the hatch door to the convex shell; and
a clasp located at a second edge of the hatch door.

20. The system of claim 10, comprising:
a cleaning cloth;
wherein the cleaning cloth comprises silver particles.
